(19) United States
(12) Patent Application Publication
(10) Pub. No.: US 2024/0041652 A1
(43) Pub. Date: Feb. 8, 2024

(54) METHODS, SYSTEMS, DEVICES FOR TREATMENTS OF RESIDUAL ASTIGMATISM, HIGH-ORDER ABERRATIONS, AND PRESBYOPIA IN HUMAN EYES

(71) Applicant: Junzhong Liang, Fremont, CA (US)

(72) Inventor: Junzhong Liang, Fremont, CA (US)

(21) Appl. No.: 18/254,243

(22) PCT Filed: Jul. 6, 2021
(86) PCT No.: PCT/US21/40513

§ 371 Date: May 24, 2023

Related U.S. Application Data
(60) Provisional application No. 63/204,483, filed on Oct. 6, 2020.
(60) Provisional application No. 63/102,887, filed on Jul. 9, 2020.

(51) Int. Cl. A61F 9/008; A61F 2/14; G02C 7/02; G02C 7/04

(52) U.S. Cl. CPC A61F 9/008; A61F 2/14; G02C 7/027; G02C 7/028; G02C 7/042; A61F 2009/0088; A61F 2240/002; G02C 2202/22

(57) ABSTRACT

Methods, systems, and devices are provided for wavefront vision correction beyond a spherocylindrical correction. Wavefront customization involves: 1) measuring wave aberration of an individual eye, 2) determining a spherocylindrical correction from the measured wavefront and a deficiency factor for the determined spherocylindrical correction, 3) inducing spherical aberration into eye's central pupil to mitigate residual aberrations beyond the spherocylindrical correction. Wavefront-optimized vision corrections can be applied to contact lenses, implantable contact lenses, intraocular lenses (IDLs), phakic IDLs, and laser vision corrections.

### RELATED APPLICATIONS

This application claims priority to U.S. provisional applications S/Ns 63/102,887 filed on Jul. 9, 2020 entitled “Methods and systems for treatment of astigmatism, coma, and high-order aberrations in human eyes” and 63/204,483 filed on Oct. 6 2020, entitled “Methods and systems for treatment of astigmatism, coma, and high-order aberrations in human eyes,” the subject matters of which are incorporated herein by reference in their entirety to the fullest extent allowed by law.

### FIELD OF THE INVENTION

Aspects and embodiments disclosed herein and claimed pertain to systems, methods, devices for refractive correction of human eyes including correction of astigmatism, high-order aberrations, and presbyopia.

### BACKGROUND

Conventional refractive corrections for human eyes over the last century provide for spherocylindrical corrections that includes a focus error (myopia and hyperopia), and astigmatim (a cylinder error). It is well know that conventional spherocylinderical correction left a host of residual high-order aberrations such as spherical aberration and coma uncorrected, and these high-order aberrations degrade acuity and quality of vision.

Introduction of modern wavefront sensor for the eye, described in “Objective measurement of wave aberrations of the human eye with the use of a Hartmann—Shack wave-front sensor” in Journal of the Optical Society of America A Vol. 11, Issue 7, (1994) pp. 1949-1957 by J Liana, B Grimm, S Goelz, and J F Bille, made it possible to precisely measure eye's aberrations using commercial aberrometers in clinical practices.

Wavefront-guided LAS IK based on wavefront aberrometers was FDA approved in the US in 2004. Although wavefront-guided LASIK was reported to be more efficacious than conventional LAS IK based on manifest refraction of a spherocylinderical correction in “Improved contrast sensitivity and visual acuity after wavefront-guided laser in situ keratomileusis: In-depth statistical analysis,” in Journal of Cataract and Refractive Surgery Volume 32, Issue 2, February (2006), pp. 215-220 by K A Tuan and J Liang, there was lacking evidence showing that high-order aberrations in human eyes were effectively eliminated by wavefront-guided LASIK. Improved vision by wavefront-guided LASIK could be attributed to a better correction for the eye's astigmatism, that can be precisely measured by waveront aberometers and corrected by surgical lasers. Effectiveness of wavefront-guided LASIK in correcting high-order aberrations is limited for at least two reasons: 1) errors in wavefront registration between wave aberration measured by wavefront aberrometers and its corresponding ablation map generated by surgical lasers, 2) effectiveness of surgical lasers in generating precise wavefront corrections towards a biological optics of human eyes.

Coma and other high-order aberrations are not correctable by spectacle eyeglasses, contact lenses, or 10Ls because 1) precise registration of a wavefront map of an eye with a spectacle lens is impossible if the eye changes its view direction through the lens, 2) contact lenses can rotate their orientation on the cornea or move their position from time to time on the cornea, 3) wavefront registration with custom-manufactured 10Ls due to corneal aberrations is almost impossible.

Consequently, although many configurations and methods for vision correction are known in the art, these conventional methods and systems suffer from one or more disadvantages as outlined above. Aspects and embodiments disclosed and claimed herein provide solutions to these disadvantageous problems.

### SUMMARY

In some embodiments, we provide a system for designing wavefront-engineered corrections for human eyes beyond a conventional spherocylindrical correction, comprising: an input module for obtaining wave aberration measurements of an eye; a processor module for i) determining a spherocylindrical correction, wherein the spherocylindrical correction consists of a focus error SPH and/or astigmatism specified by CYL and AXIS, ii) determining a deficiency factor for the spherocylindrical correction, wherein the deficiency factor includes degraded best corrected acuity and/or degraded quality of vision due to uncorrectable astigmatism, coma, and other high-order aberrations in the eye, iii) determining at least a wavefront component covering the central pupil of an eye up to 4.5 mm in diameter, wherein the wavefront component induces additional spherical aberration into the corrected eye for mitigating residual refractive errors beyond the spherocylindrical correction; an output module for communicating the spherocylindrical correction as well as the designed wavefront component covering the central pupil of the eye for at least one optical design for the optimized vision correction beyond a spherocylindrical correction.

In other embodiments, we provide a wavefront-engineered ophthalmic lens, configured as an implantable lens or wearable lens, comprising: an optic having an anterior surface and a posterior surface; the optic refracting light in an optical section having a diameter up to 8 mm and configured into a plurality of optical sections, wherein: i) in an inner central optical section with a diameter of typical 3 mm or between 2.5 mm and 4.5 mm, the optic is configured to induce additional spherical aberration for treatment of uncorrected refractive errors including residual astigmatism, coma, and other high-order aberrations, presbyopia in the eye left by the spherocylindrical correction, wherein the induced spherical aberration includes a positive spherical aberration, a negative spherical aberration, spherical aberrations of opposite sign, ii) the optic has a baseline extending across the entire optical section for the correction of a spherocylindrical correction.

In yet other embodiments, we provide an intraocular lens, comprising: a lens having an anterior surface and a posterior surface; a diffractive profile disposed on one of the anterior surface and the posterior surface, the diffractive profile comprising a plurality of concentric zones configured to produce constructive interference in a plurality of diffractive orders within a range of vision; and an aspherical profile disposed on the anterior surface or the posterior surface without the diffractive profile in the central portion having a diameter up to 4.5 mm, wherein the aspherical profile induces spherical aberration into the eye's central pupil for treatment of uncorrected residual refractive errors in the eye left by the spherocylindrical correction and/or for extending depth of focus for images of the diffractive orders.

In still other embodiments, we provide an ophthalmic lens, comprising: an optic having an anterior surface and a posterior surface disposed about an optical axis; wherein at least one of the surfaces has a profile characterized by superposition of a base profile and two auxiliary profiles, and the auxiliary profiles are distributed over a plurality of concentric zones in the central portion of the lens, further wherein the baseline profile defines a monofocal lens if the auxiliary profiles are absent; the central concentric zones have a central circular zone with radius of r₁ and a plurality of annular zones with outer radius of rₙ (r₂ for the first annular zone and r3 for the second annular zone, and so on); the first auxiliary profiles are expressed by f₁(r) cos[2π²/t₁(r)] for the central circular zone, −f₂(r) cos[2π(r−r₁)²/t₂(r)] for the first annular zone, f₃(r) cos[2π(r−r₂)²/t₃(r)] for the next annular zone and so on, wherein fₙ(r), including f₁(r), f₂(r), f₃(r), are slow changing functions for amplitude modulation while tₙ(r), including t₁(r), t₂(r), t₃(r), are variables for frequency modulation; and the second auxiliary profile provides focus shift(s) in at least one of the concentric zones.

### BRIEF DESCRIPTION OF THE DRAWINGS

FIG. 1 shows calculated Point-Spread-Functions (PSFs) and calclated retinal images of an acuity chart under a spherocylindrical correction for four human eyes. Three pupil sizes are considered: 3 mm, 3.5 mm, and 4.5 mm.

FIG. 2 shows calcuated Modulation-Transfer-Functions (MTFs) of the same four eyes in FIG. 1 for a 3 mm pupil in comparison to the population mean MTF of normal human eyes as well as a mean MTF of post-op eyes of 20 subjects with monofocal Intraocular Lenses (IDLs).

FIG. 3 shows calculated PSFs, retinal images of an acuity chart, and MTFs of an eye with significant uncorrected aberrations. Refractive correction of the eye with a standard lens that provides a spherocylinder correction is compared to two wavefront designs that induces additional spherical aberration into a 3.5 mm pupil in addition to a spherocylinder correction. Three pupil sizes are considered: 3 mm, 3.5 mm, and 4.5 mm.

FIG. 4 shows calculated PSFs, retinal images of an acuity chart, and MTFs of another eye with significant uncorrected aberrations. Refractive correction of the eye with a spherocylinder correction is compared to two wavefront designs that induces additional spherical aberration into a 3.5 mm pupil in addition to a spherocylinder correction.

FIG. 5 shows modules of a system for optimizing vision correction of an eye beyond a spherocylindrical correction in one exemplary aspect.

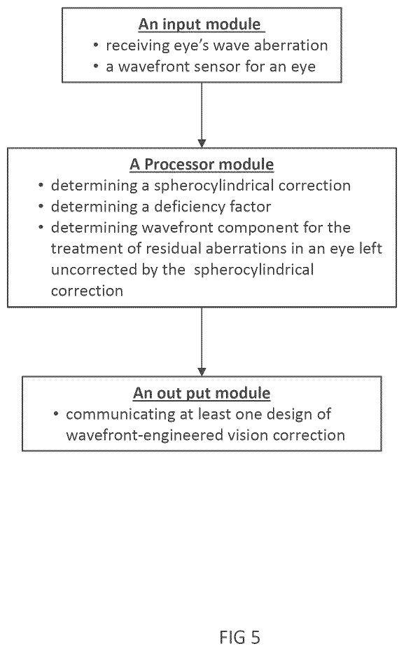

FIG. 6A shows calculated retinal images of an acuity chart for an eye with significant uncorrected aberrations. Refractive correction of the eye with a standard lens, which provides a spherocylinder correction, is compared to two wavefront designs that induces spherical aberrations of opposite sign in addition to a spherocylindrical correction.

FIG. 6B shows calculated retinal images of an acuity chart for another eye with significant uncorrected aberrations. Refractive correction of the eye with a standard lens is compared to two wavefront designs that induces spherical aberrations of opposite sign in addition to a spherocylindrical correction.

FIG. 7 shows a schematic diagram of an ophthalmic lens for an eye having an aspherical section that induces spherical aberrations of opposite sign into the eye's central pupil for treatment of uncorrected residual refractive errors beyond a spherocylindrical correction.

### DETAILED DESCRIPTION

Inducing spherical aberration into an eye's central pupil to mitigate astigmatism and coma was disclosed in PCT/US2020/027548 by J Liang and L. Yu. To be effective in correcting eye's aberrations as well as effectively prescribing the related lenses must take into account not only the complexity of the eye's high-order aberrations but also any interaction between residual astigmatism and high-order aberrations in human eyes. Eye's aberrations are known to be different from eye to eye, and as many as 65 Zernike polynomials were used to describe eye's wave aberrations in “Aberrations and retinal image quality of the normal human eye” published in Journal of the Optical Society of America A Vol 14, Issue 11, pp. 2873-2883 (1997)) by J Liang and D. R. Williams.

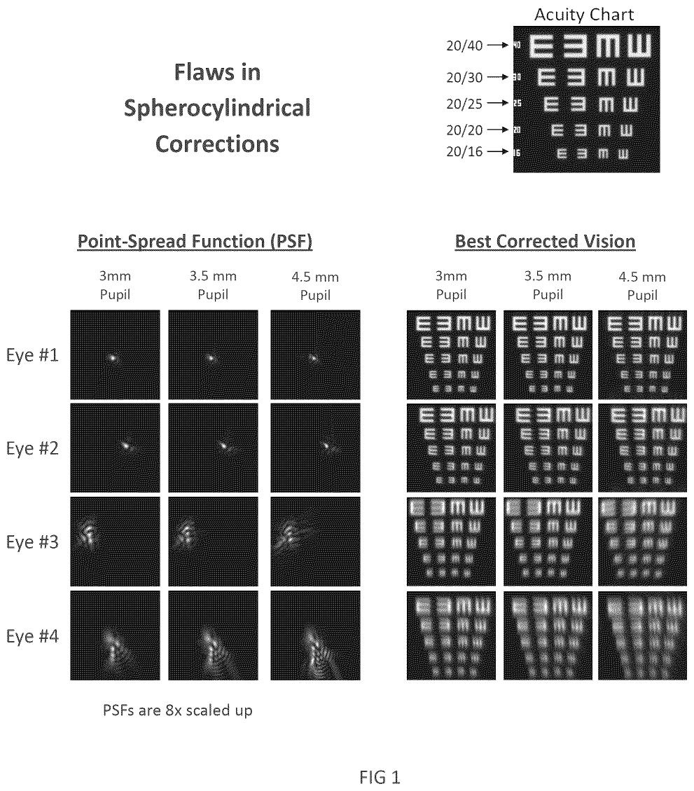

FIG. 1 shows calculated Point-Spread-Functions (PSFs) and retinal images of an acuity chart under a spherocylindrical correction for four eyes. Wave aberration measurements of these four eyes were obtained with a Hartmann-Shack sensor that samples wavefront slope every 0.15 mm across eye's pupil, and 65 Zernike polynomials were also used to describe eye's wave aberrations. A spherocylindrical correction is determined for each eye for eyeglasses, contact lenses, or Intra-ocular lenses based on two conditions: 1) astigmatism (CYL) is corrected if it is equal to or more than 0.5D and with an incremental step of 0.25D; 2) Focus error (SPH) is corrected with a incremental step of 0.12D; 3) high-order aberrations including coma, spherical aberration, and trefoil are left uncorrected. Three pupil sizes are considered: 3 mm, 3.5 mm, and 4.5 mm.

It is not difficult to realize the following from FIG. 1: 1) not all eyes are the same, 2) eyes with poor optical quality (Eye#3 and Eye#4) are either limited for below 20/20 acuity, or having poor quality of vision (distorted letters) even if 20/20 can be achieved, 3) treatments for uncorrected aberrations in Eye#3 and Eye#4 are unmet needs in refractive corrections. Blurred vision (Horizontal Es in acuity chart) for Eye#3 and Eye#4 in comparison to relatively clear vision for Eye#1 and Eye#2 can be easily explained by differences in their corresponding PSFs. PSFs of Eye#4 and Eye#3 are wide with complicated structures and shapes, while the PSFs for Eye#1 and Eye#2 are more compact and smaller. It must be mentioned that the PSFs, compared to the acuity chart, are scaled up eight (8) times in dimension in order to show fine structures of eye's PSFs.

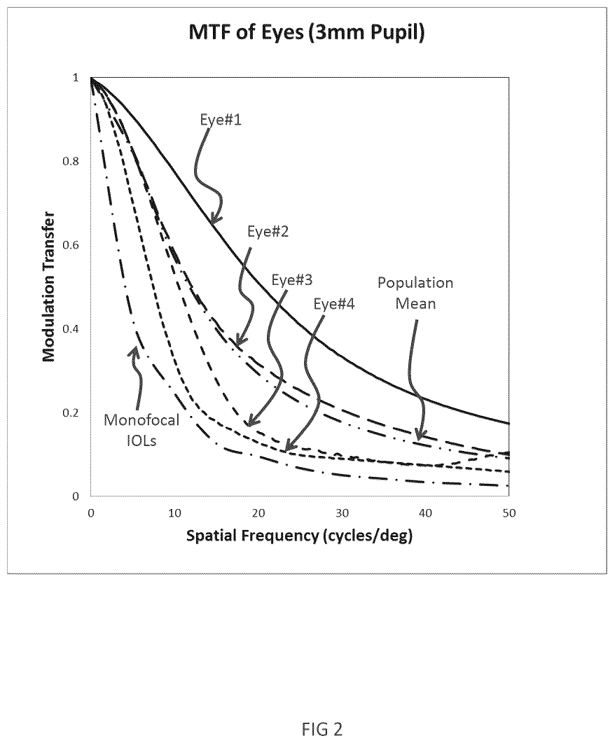

FIG. 2 shows calcuated Modulation-Transfer-Functions (MTFs) of the same four eyes in FIG. 1 for a 3 mm pupil in comparision to 1) a mean MTF of normal human eyes according to “a formula for the mean human optical modulation transfer (Journal of Vision (2013) 13(6): 18, 1-11, by A. B. Watson),” and 2) a mean MTF of post-op eyes with monofocal Intraocular Lenses (IOLs) from “Corneal optical aberrations and Retinal image quality in patients in whom monofocal intraocular Lenses were implanted” (Arch Ophthamology, (2002), 120: 1143-1151 by A. Guirao et al).

Based on eye's MTF, three classes of optical quality can be used for characterizing human eyes: 1) Class I optics with its MTF far above the population mean MTF for Eye#1, 2) Class II optics with its MTF about the population mean MTF for Eye#2, 3) Class III optics with its MTF far below the population mean MTF for Eye#3 and Eye#4.

It must be emphasized that the mean MTF of the normal population in the literature was derived with a perfect correction of astigmatism in the human eyes, which is rarely achieved in conventional spherocylndrical corrections with eyeglasses, contact lenses, and IDLs. If uncorrected astigmatism in eye is taken into account, mean MTF of normal human eyes under a spherocylinderical correction will be far below the curve in FIG. 2.

Additionally, it is also clearly seen that MTF of eyes with monofocal IDLs is not only below the mean MTF of normal human eyes but also lower than MTFs of Eye#3 and Eye#4. Therefore, mitigation of residual aberrations for post-op IOL eyes is also an unmet medical need for improving acuity and quality of vision.

In some embodiments of present inventions, we disclose a wavefront method for treatment of eye's aberrations beyond a spherocylindrical correction. The method comprises the steps of: 1) measuring wave aberration of an eye, 2) determining a spherocylindrical correction from the measured wave aberration, 3) determining at least a wavefront component covering central pupil of an eye having a diameter about 3 mm (more than 2.5 mm and less than 4.6 mm). The wavefront component induces additional spherical aberration into the corrected eye for mitigating residual refractive errors beyond the determined spherocylindrical correction, which include but are not limited to coma and residual astigmatism.

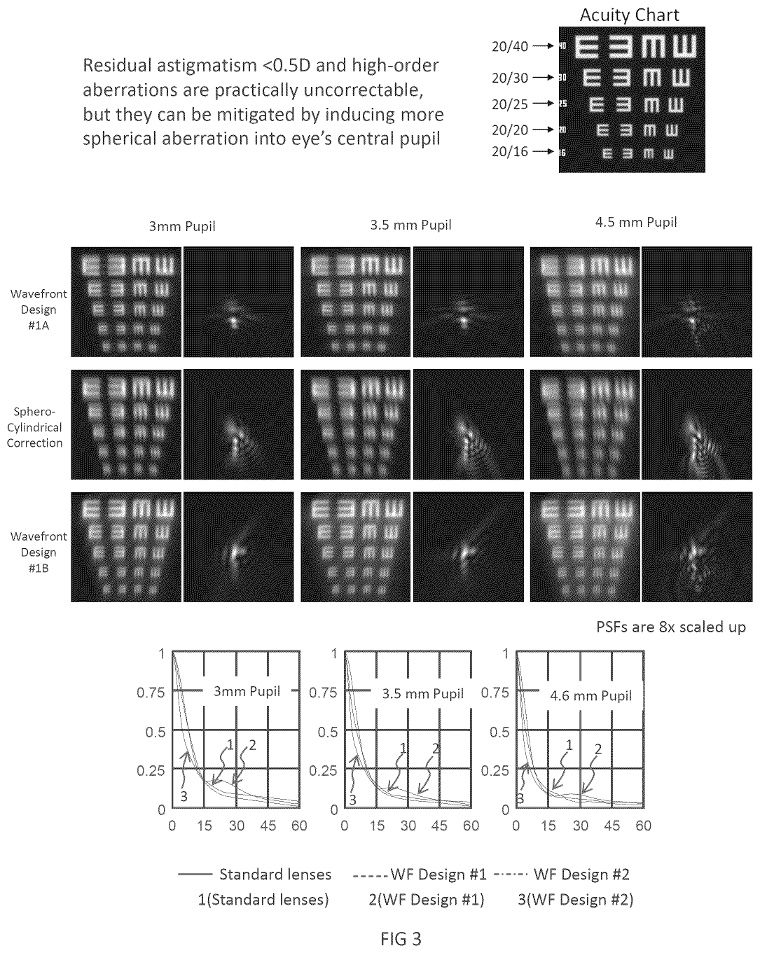

With the aberrations in Eye#4 known from an wavefront aberrometer, we determined 1) a controlled amount of negative spherical aberration in wavefront design #1 and a controlled amount of positive spherical aberration in wavefront design #2 both within a 3.5 mm pupil, 2) a SPH offset from the determined spherocylindrical correction. It must be emphasized that the controlled amount of negative/positive positive spherical aberration in design #1/#2 as well as the SPH offset must be custom determined based on residual aberrations in the eye. The two wavefront designs are compared with the conventional sphero-cylidrical correction in FIG. 3 for Eye#4, showing calculated PSFs, retinal images of an acuity chart, and MTFs for three pupil sizes: 3 mm, 3.5 mm, and 4.5 mm.

As shown in FIG. 3, even though the residual aberrations in Eye#4 are not correctable, they can be mitigated by inducing more spherical aberration into eye's central pupil with the two wavefront designs. Wavefront corrections provide: 1) improved acuity of 20/16, 2) significantly better night vision, 3) better quality of vision by eliminating image distortion.

It must be noticed that inducing additional spherical aberration into eye's central pupil does not lead to MTF reduction. Instead, eye's point-spread image is simply reshaped, leading to effective mitigation to uncorrectable aberrations in the eye.

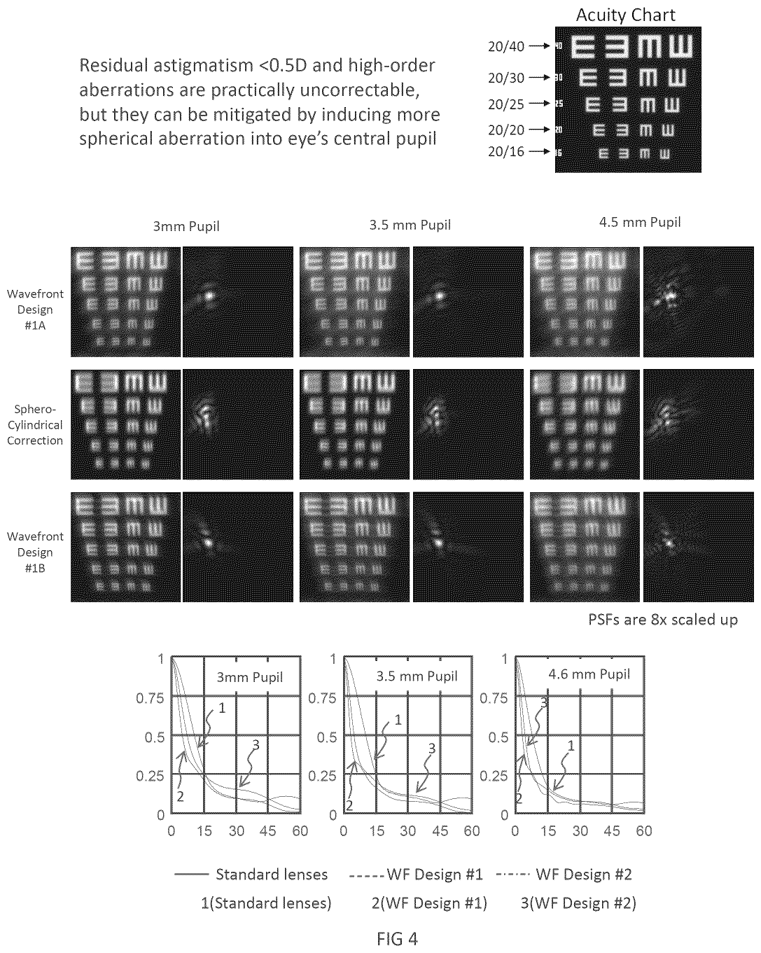

The same wavefront optimization was applied to Eye#3 with results in FIG. 4, showing the calculated PSFs, retinal images of an acuity chart, and MTFs. Similar conclusions are obtained for Eye#3: even though the residual aberrations are not correctable, they can be mitigated by inducing more spherical aberration into eye's central pupil with a 3.5 mm pupil in the two wavefront designs. It must be emphasized that the controlled amount of negative/positive positive spherical aberration in design #1/#2 as well as the SPH offset must be custom determined based on residual aberrations in the eye. Additional advantages of our wavefront treatments include: 1) tolerance in

lens rotation because both spherical aberration and focus offset is rotationally symmetric, 2) tolerance of lens displacements as much as 0.75 mm according to our simulation, 3) tolerance in manufacturing errors in SPH/CYL.

In one embodiment, the method in the present invention for wavefront-engineered corrections by inducing spherical aberration into eye's central pupil further includes prescribing a contact lens, a surgical procedure such as a laser vision correction, or surgical implants of a phakic IOL.

In another embodiment, the induced spherical aberration into eye's central pupil includes: i) a positive spherical aberration, ii) a negative spherical aberration, iii) spherical aberrations of opposite sign in two concentric zones.

In some embodiments, we disclose a system for designing wavefront-engineered corrections of human eyes beyond a spherocylindrical correction. The system comprises 1) an input module for obtaining wave aberration of an eye, 2) a processor module for i) determining a spherocylindrical correction that consists of a focus error SPH and/or astigmatism specified by CYL and AXIS, ii) determining a deficiency factor for the spherocylindrical correction, and the deficiency factor includes degraded best corrected acuity and/or degraded quality of vision due to residual astigmatism, coma, and other high-order aberrations in the eye, iii) determining at least a wavefront component covering central pupil of an eye having a diameter of about 3.5 mm up to 4.5 mm, 3) an output module for communicating the spherocylindrical correction as well as the designed wavefront component covering central pupil of the eye for at least one optical design for the optimized vision correction beyond a spherocylindrical correction. The wavefront component induces additional spherical aberration into the corrected eye for mitigating residual refractive errors beyond the spherocylindrical correction.

In one embodiment, determining a deficiency factor for the spherocylindrical correction includes providing at least one simulated retinal image of an acuity chart under the spherocylindrical correction and estimating a best corrected acuity. For eyes with significant uncorrected aberrations, the estimated acuity would be worse than 20/20.

In another embodiment, determining a deficiency factor for the spherocylindrical correction includes i) calculating optical quality of an eye from the residual aberration under a spherocylindrical correction, ii) comparing the calculated optical quality of the eye with a defined metrics from normal human eyes and determining necessity of inducing additional spherical aberration into eye beyond the spherocylindrical correction. The residual aberration is the difference between the wave aberration of an eye and the spherocylindrical correction. The optical quality can be modulation transfer function (MTF) and the defined metrics from normal human eyes can be a mean MTF from a normal population.

In yet another embodiment, determining a deficiency factor for the spherocylindrical correction includes receiving a desired presbyopia power for a presbyopia correction beyond the spherocylindrical correction. The presbyopia power is positive and between +0.5D and +3.5D.

In one embodiment, communicating at least one optical design for the optimized wavefront correction further includes showing vision results from a plurality of design options so that the best correction can be selected manually.

In another embodiment, the optimized vision correction is applied to an ophthalmic lens having an optical section with a diameter up to 8 mm. The ophthalmic lens includes a contact lens, an implantable contact lens (ICL), an intraocular lens (IOL), a phakic IOL, and an accommodating IOL.

In yet another embodiment, the optimized vision correction is further applied to a laser vision correction.

In one embodiment, the input module is a wavefront sensor for an eye that provides measurements of eye's wave aberration, or it receives eye's wave aberration from another device such as a wavefront sensor for an eye.

In another embodiment, inducing additional spherical aberration into the corrected eye includes: i) a positive spherical aberration, ii) a negative spherical aberration, iii) spherical aberrations of opposite sign in two concentric zones.

In yet another embodiment, the output module includes i) a display device, ii) generating a file that can be transmitted to another display device.

In still another embodiment, the system for optimizing vision correction of an eye beyond a spherocylindrical correction further includes a phoropter module for updating the determined spherocylindrical correction.

Wavefront-engineered multifocal lenses is disclosed in US provisional patents (#62/920,859, #62/974,317, #62/995/872) for treatment of presbyopia in addition to a spherocylindrical correction.

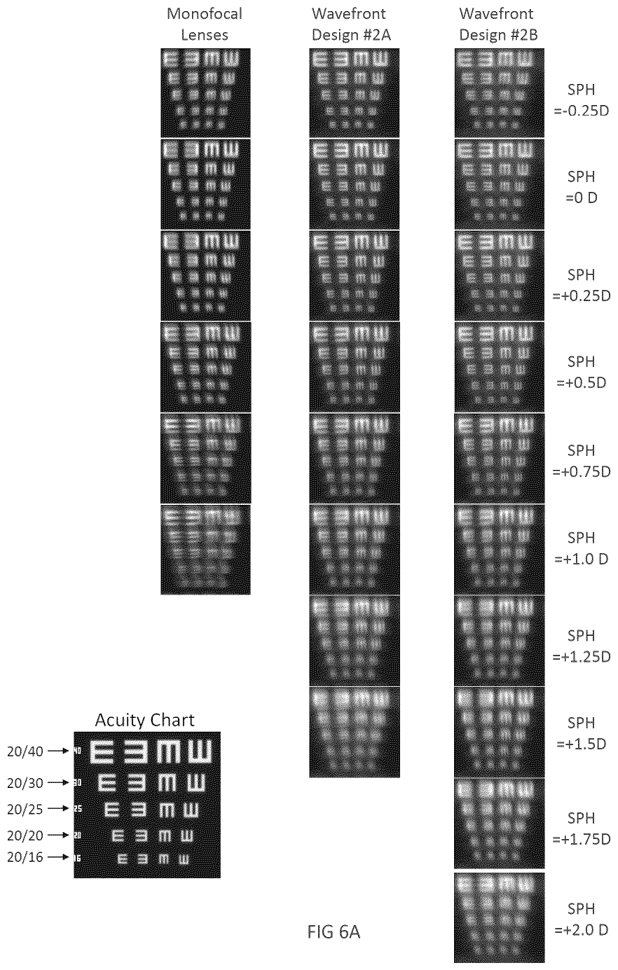

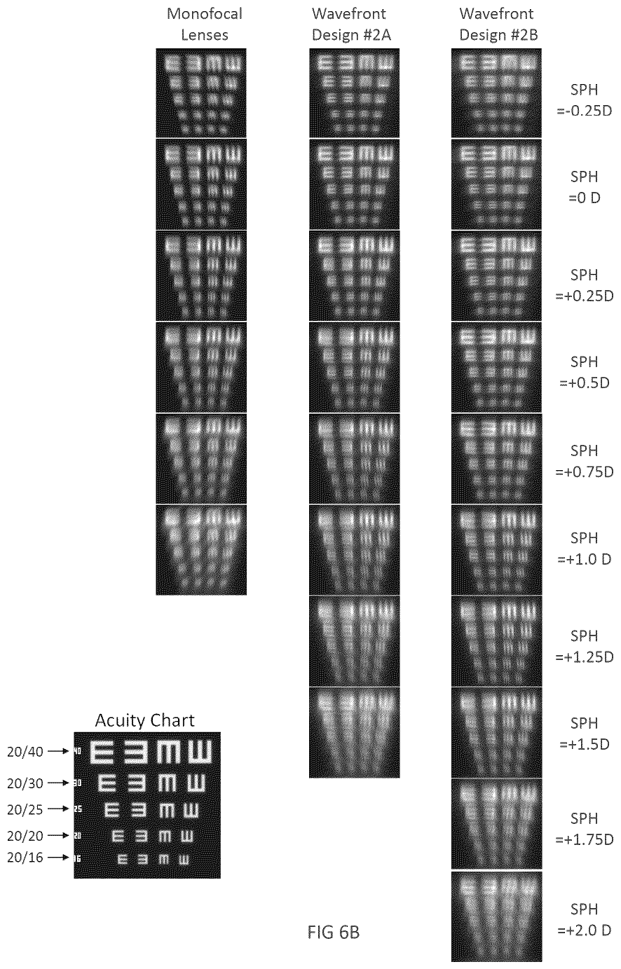

We applied two designs of wavefront multifocal lenses (Wavefront Design #2A and Wavefront Design #2B) to eyes with Class 3 optics (Eye#3 and Eye#4). FIG. 6A shows calculated retinal images of an acuity chart for Eye#3 for a pupil size of 3 mm. Monofocal lenses with a standard spherocylindrical correction are compared with wavefront multifocal lenses (Wavefront Design #2A and Wavefront Design 2B). The designs involve inducing spherical aberrations of opposite sign into central pupil of eye. Retinal images for an entire focus range from −0.25D to +2.0D are compared side by side.

It is seen in FIG. 6A that the wavefront multifocal lenses not only improve depth of focus for a presbyopia correction but also mitigate residual aberrations left uncorrected by conventional spherocylindrical correction, leading to improved acuity of 20/16 while the monofocal lens with a spherocylindrical correction has worse than 20/20. The same result was found for Eye#3 in FIG. 6B, showing calculated retinal images of an acuity chart with significant uncorrected aberrations trough for a pupil size of 3 mm.

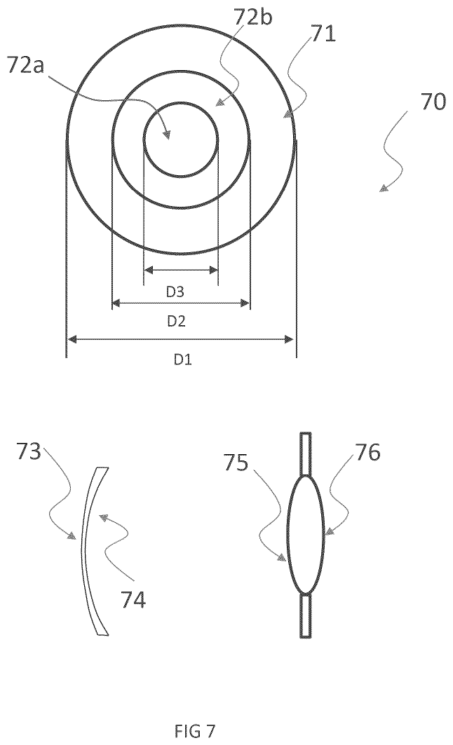

In some embodiments we describe a wavefront-engineered ophthalmic lens that can be configured as an implantable lens or a wearable lens. FIG. 7 shows a schematic diagram of an ophthalmic lens 70. The lens comprises an optic having an anterior surface 73 and 75 and a posterior surface 74 and 76, the optic refracts light in an optical section having a diameter D₁ up to 8 mm which is configured into a plurality of optical sections. In addition to a baseline Diopter that extends across the entire optical section (71, 72a, 72b) for the correction of a spherocylindrical correction, the optic in an inner central optical section 72a and 72b with a diameter D2 of typically 3 mm or between 2.5 mm and 4.5 mm is configured to induce additional spherical aberration for treatment of uncorrected refractive errors in the eye left by the spherocylindrical correction, which include residual astigmatism, coma, and other high-order aberrations. The induced spherical aberration includes a positive spherical aberration, a negative spherical aberration. In one embodiment, inducing additional spherical aberration in the inner central optical section is achieved using aspherical surfaces.

In one example in FIG. 7, the wavefront (aspherical) section induces a positive spherical aberration in the central zone with a diameter of D3, and a negative spherical aberration in the annular zone with an outer diameter of D2.

In one embodiment, the wavefront-engineered ophthalmic lens is further configured to be a contact lens, an intraocular lens (IOL), a phakic IOL, or an implantable contact lens.

In still some embodiments of present inventions, we disclose a contact lens for vision tests of human eyes. The lens comprises of an optic having an anterior surface and a posterior surface and the optic refracts light in an optical section having a diameter up to 8 mm. In addition, optical section of the lens is configured into a plurality of optical sections: I) in an outer annular optical section the optic is a monofocal lens or a powerless plate, II) in an inner central optical section, located inside the outer annular optical section, with a diameter of typical 3 mm or between 2.5 mm and 4.5 mm, the optic is configured to induce additional spherical aberration into eye's central pupil in one of the following forms: i) a positive spherical aberration, ii) a negative spherical aberration, iii) spherical aberrations of opposite sign.

In one embodiment, the contact lens for vision tests of human eyes further includes a focus power and/or a cylinder power.

In another embodiment, the outer annular optical section of the contact lens for vision tests of human eyes has reduced light transparency, which can be tinted or printed.

In an embodiment, we disclose an intraocular lens that comprises: 1) a lens having an anterior surface and a posterior surface, 2) a diffractive profile is disposed on one of the anterior surface and the posterior surface, and the diffractive profile comprises a plurality of concentric zones configured to produce constructive interference in a plurality of diffractive orders within a range of vision, 3) an aspherical profile is disposed on the anterior surface or the posterior surface without the diffractive profile having a diameter up to 4.5 mm. The aspherical profile in the central portion of the lens induces spherical aberration into the eye's central pupil for treatments of potential residual refractive errors in the eye left by the spherocylindrical correction and/or for extending depth of focus for the images associated with the diffractive orders.

The potential uncorrected residual refractive errors include astigmatism, coma, and high-order aberrations while the induced spherical aberration by the aspherical profile is represented by one of the following forms: i) a positive spherical aberration, ii) a negative spherical aberration, iii) spherical aberrations of opposite sign.

In another embodiments, we disclose another ophthalmic lens, and it comprises: 1) an optic having an anterior surface and a posterior surface disposed about an optical axis, 2) at least one of the surfaces has a profile characterized by superposition of a base profile and two auxiliary profiles. The auxiliary profiles are distributed over a plurality of concentric zones in the central portion of the lens while the baseline profile, covering the entire lens, defines a monofocal lens if the auxiliary profiles are absent. The central concentric zones have a circular area with radius of r1 and a plurality of annular zones with outer radius of rn (r2 for the first annular zone and r3 for the second annular zone, and so on). The first auxiliary profiles can be expressed by f1(r) cos[2π2/t1(r)] for the central circular zone, −f2(r) cos[2π(r−r1)2/t2(r)] for the first annular zone, f3(r) cos[2π(r−r2) 2/t3(r)] for the next annular zone and so on. The functions fn(r), including f1(r), f2(r), f3(r), are slow changing for amplitude modulation while tn(r), including t1(r) t2(r) t3(r), are variables for frequency modulation. The second auxiliary profile provides focus shift(s) in at least one of the concentric zones.

Three advanced features are introduced to overcome limitations associate with modulated periodical lenses described by Xin Hong in U.S. Pat. No. 9,101,466 B2.

In one aspect, if fn(r) and Tn(r) form their own continues function like those in U.S. Pat. No. 9,101,466 B2, the second auxiliary profile can provide an independent focus control needed in the central circular zone. A controlled focus offset in the central circular zone is important for controlling the total depth of focus from the DISTANCE focus to the NEAR focus, which can be found with the wavefront bifocal, trifocal lenses in the PCT application #PCT/US2020/027548, titled “METHODS AND DEVICES FOR WAVEFRONT TREATMENTS OF ASTIGMATISM, COMA, PRESBYOPIA IN HUMAN EYES” by J. Liang and L. Yu. It is also evident that two different focus offsets are used for optimizing the lenses with two aspherical zones.

In another aspect, the discrete functions fn(r) and Tn(r) allow to control focus error, primary spherical aberration, and high-order spherical aberrations for each aspherical zone.

In one example, if both fn(r) and Tn(r) are constant, the first auxiliary

profile for the n-th zone can be expressed as

$$\begin{matrix}{W}_{n} ( r ) = \pm {f}_{n} ( r ) cos [ 2 π {r}^{2} / {t}_{n} ( r ) ] \\ = \pm c * cos ( 2 π {r}^{2} / T )\end{matrix}$$

where r is the radial distance from the lens center, c and T are both constants. The Taylor expansion of Wn(r) is

$$\begin{matrix}{W}_{n} ( r ) = \pm c * cos ( 2 π {r}^{2} / T ) \\ = \pm c * [ 1 - {( 2 π {r}^{2} / T )}^{2} / 2 ! + {( 2 π {r}^{2} / T )}^{4} / 4 ! - {( 2 π {r}^{2} / T )}^{6} / 6 ! + … ] \\ = \pm ( c - {c}_{1} * {r}^{4} + {c}_{2} * {r}^{8} - {c}_{3} * {r}^{12} + … )\end{matrix} \qquad ( 1 )$$

where c, c1, c2, c3 are constants. Except for the constant term (c), the auxiliary profile induces primary spherical aberration r4 as well as high-order spherical aberrations (r8 ,r12 and etc). It is also noticed that the coefficient for spherical aberrations (c1, c2, c3) are related.

In another example, if fn(r) and Tn(r) in any section are controlled so that the first auxiliary profile for the n-th zone can be approximated by a Gaussian function

$$\begin{matrix}{W}_{n} ( r ) = {f}_{n} ( r ) cos [ 2 π {r}^{2} / {t}_{n} ( r ) ] \\ \approx \pm q * exp ( - {dr}^{2} )\end{matrix}$$

where r is the radial distance from the lens center, q and d are both constants. The Taylor expansion of Wn(r) is

$$\begin{matrix}{W}_{n} ( r ) = \pm q * exp ( - {dr}^{2} ) \\ = \pm q * [ 1 + ( - {dr}^{2} ) + {( - {dr}^{2} )}^{2} / 2 ! + {( - {dr}^{2} )}^{3} / 3 ! + {( - {dr}^{2} )}^{4} / 4 ! + … ] \\ = \pm [ q - q * d * {r}^{2} + ( q * {d}^{2} / 2 ) {r}^{4} - ( q * {d}^{3} / 6 ) {r}^{6} + \\ ( q * {d}^{4} / 24 ) {r}^{8} + … ] \\ = \pm ( q - {q}_{1} * {r}^{2} + {q}_{2} * {r}^{4} - {q}_{3} * {r}^{6} + {q}_{4} * {r}^{8} + … )\end{matrix} \qquad ( 2 )$$

Except for the constant term, the auxiliary profile induces a focus offset term (r2), a primary spherical aberration term r4 as well as high-order spherical aberration terms(r6 ,r8). It is also noticed that the coefficients for the focus offset (q1) and spherical aberrations (q2, q3, q4) are related.

In yet another aspect, individual functions fn(r), including f1(r), f2(r), f3(r), provides the freedom beyond a typical cosine function for the amplitude modulation in U.S. Pat. No. 9,101,466 B2. This allows controls of 1) total number of aspheric zones and 2) relative modulation strength between the zones.

In one embodiment, the second auxiliary profile provides a focus shift in central circular zone. In another embodiment, the ophthalmic lens is further configured to be a contact lens, an intraocular lens (IOL), a phakic IOL or an implantable contact lens.

What is claimed is:

1. A system for designing wavefront-engineered corrections for human eyes beyond a conventional spherocylindrical correction, comprising:
an input module adapted to obtain a wave aberration of an eye;
a processor module adapted to i) determine a spherocylindrical correction, wherein the spherocylindrical correction consists of a focus error SPH and/or astigmatism specified by CYL and AXIS, ii) determine a deficiency factor for the spherocylindrical correction, wherein the deficiency factor includes a degraded best corrected acuity and/or degraded quality of vision due to uncorrectable astigmatism, coma, and other high-order aberrations in the eye, iii) determine at least a wavefront component covering a central pupil of an eye up to 4.5 mm in diameter, wherein the wavefront component induces additional spherical aberration into the corrected eye for mitigating residual refractive errors beyond the spherocylindrical correction; and
an output module adapted to communicate the spherocylindrical correction as well as the designed wavefront component covering the central pupil of the eye for at least one optical design for the optimized vision correction beyond a spherocylindrical correction.

2. The system of claim 1, wherein determining a deficiency factor for the spherocylindrical correction includes providing at least one simulated retinal image of an acuity chart under the spherocylindrical correction and estimating a best corrected acuity.

3. The system of claim 1, wherein determining a deficiency factor for the spherocylindrical correction includes i) calculating optical quality of an eye from the residual aberration under a spherocylindrical correction, wherein the residual aberration is the difference between the wave aberration of an eye and the spherocylindrical correction, ii) comparing the calculated optical quality of the eye with a defined metrics from normal human eyes and determining necessity of inducing additional spherical aberration into eye beyond the spherocylindrical correction.

4. The system of claim 3, wherein the optical quality is modulation transfer function (MTF) and the defined metrics from normal human eyes is a mean MTF from a normal population.

5. The system of claim 1, wherein determining a deficiency factor for the spherocylindrical correction includes receiving a desired presbyopia power for a presbyopia correction beyond the spherocylindrical correction, wherein presbyopia power is positive between +0.5D and +3.5D.

6. The system of claim 1, wherein communicating at least one optical design for the optimized vision correction further includes showing a plurality of design options so that the best correction can be selected.

7. The system of claim 1, wherein the optimized vision correction is further applied to an ophthalmic lens having an optical section with a diameter up to 8mm, wherein the ophthalmic lens includes a contact lens, an implantable contact lens (ICL), an intraocular lens (IOL), a phakic IOL, and an accommodating IOL.

8. The system of claim 1, wherein the optimized vision correction is further applied to a laser vision correction.

9. The system of claim 1, wherein the input module is a wavefront sensor for an eye that provides measurements of eye's wave aberration.

10. The system of claim 1, wherein the input module receives eye's wave aberration from another device such as a wavefront sensor for an eye.

11. The system of claim 1, wherein inducing additional spherical aberration into the corrected eye includes: i) a positive spherical aberration, ii) a negative spherical aberration, iii) spherical aberrations of opposite sign in two concentric zones.

12. The system of claim 1, wherein the output module includes a display device and/or generating a file that can be transmitted to another display device.

13. The system of claim 1, further including a phoropter module for updating the determined spherocylindrical correction.

14. A method for treatment of eye's aberrations beyond a spherocylindrical correction, comprising:
measuring a wave aberration of an eye;
determining a spherocylindrical correction from the measured wave aberration;
determining at least a wavefront component covering central pupil of an eye having a diameter more than 2.5 mm and less than 4.6 mm, wherein the wavefront component induces additional spherical aberration into the corrected eye for mitigating residual refractive errors beyond the determined spherocylindrical correction, wherein inducing additional spherical aberration includes i) a positive spherical aberration, ii) a negative spherical aberration, iii) spherical aberrations of opposite sign in two concentric zones.

15. The method of claim 14, further including prescribing a contact lens, a surgical procedure such as a laser vision correction, or surgical implants of a phakic IOL.

16. A wavefront-engineered ophthalmic lens, configured as an implantable lens or wearable lens, comprising: an optic having an anterior surface and a posterior surface;
the optic refracting light in an optical section having a diameter up to 8mm and configured into a plurality of optical sections, wherein: I) in an inner central optical section with a diameter of typical 3mm or between 2.5 mm and 4.5 mm, the optic is configured to induce additional spherical aberration for treatment of uncorrected refractive errors, including residual astigmatism, coma, and other high-order aberrations, and presbyopia in the eye left by the spherocylindrical correction, wherein the induced spherical aberration includes a positive spherical aberration, a negative spherical aberration, spherical aberrations of opposite sign, II) the optic has a baseline extending across the entire optical section for the correction of a spherocylindrical correction.

17. The lens of claim 16, configured to be a contact lens, an intraocular lens (IOL), a phakic IOL, or an implantable contact lens.

18. An intraocular lens, comprising:
a lens having an anterior surface and a posterior surface;
a diffractive profile disposed on one of the anterior surface and the posterior surface, the diffractive profile comprising a plurality of concentric zones configured to produce constructive interference in a plurality of diffractive orders within a range of vision; and
an aspherical profile disposed on the anterior surface or the posterior surface without the diffractive profile in the central portion having a diameter up to 4.5 mm, wherein the aspherical profile induces spherical aberration into the eye's central pupil for treatments of uncorrected residual refractive errors in the eye left by the spherocylindrical correction and/or for extending depth of focus for images of the diffractive orders.

19. The lens of claim 18, wherein the uncorrected residual refractive errors include astigmatism, coma, and high-order aberrations.

20. The lens of claim 18, wherein the induced spherical aberration into eye's central pupil is represented by one of the following forms: i) a positive spherical aberration, ii) a negative spherical aberration, iii) spherical aberrations of opposite sign.

21. A contact lens for vision tests of human eyes, comprising:
an optic having an anterior surface and a posterior surface; the optic refracting light in an optical section having a diameter up to 8mm and configured into a plurality of optical sections, wherein: I) in an outer annular optical section the optic is a monofocal lens or a powerless optical plate, II) in an inner central optical section, located inside the outer annular optical section, with a diameter of typical 3 mm or between 2.5 mm and 4.5 mm, the optic is configured to induces additional spherical aberration into eye's central pupil in one of the following forms: i) a positive spherical aberration, ii) a negative spherical aberration, iii) spherical aberrations of opposite sign.

22. The lens of claim 21, wherein the outer annular optical section has reduced light transparency.

23. The lenses of claim 22, wherein the outer section is tinted or printed.

24. The lens of claim 21, further including a focus power and/or a cylinder power.

25. An ophthalmic lens, comprising:
an optic having an anterior surface and a posterior surface disposed about an optical axis; wherein:
at least one of the surfaces has a profile characterized by superposition of a base profile and two auxiliary profiles, and the auxiliary profiles are distributed over a plurality of concentric zones in the central portion of the lens, wherein:
the baseline profile defines a monofocal lens if the auxiliary profiles are absent;
the central concentric zones have a central circular zone with radius of r1 and a plurality of annular zones with outer radius of rₙ(r₂ for the first annular zone and r3 for the second annular zone, and so on);
the first auxiliary profiles are expressed by f₁(r) cos[2π²/t₁(r)] for the central circular zone, −f₂(r) cos[2π(r−r₁)²/t₂(r)] for the first annular zone, f₃(r) cos[2π(r−r₂)²/t₃(r)] for the next annular zone and so on, wherein fₙ(r), including f₁(r), f₂(r), f₃(r), are slow changing functions for amplitude modulation while tₙ(r), including t₁(r), t₂(r), t₃(r), are variables for frequency modulation; and
the second auxiliary profile provides focus shift(s) in at least one of the concentric zones.

26. The lens of claim 25, wherein the second auxiliary profile provides a focus shift in central circular zone.

27. The lens of claim 25, further configured to be a contact lens, an intraocular lens (IOL), a phakic IOL, or an implantable contact lens.
